(19) United States
(12) Patent Application Publication
(10) Pub. No.: US 2024/0115497 A1
(43) Pub. Date: Apr. 11, 2024

(54) INHALABLE PHARMACEUTICAL POWDER FORMULATION AND PREPARATION METHOD THEREFOR

(71) Applicant: ZHEJIANG CUIZE PHARMACEUTICAL TECHNOLOGY CO., LTD., Hangzhou,Zhejiang (CN)

(72) Inventors: Siping SUN, Hangzhou (CN); Shaojun FENG, Hangzhou (CN); Qiandao GU, Hangzhou (CN)

(21) Appl. No.: 18/264,327

(22) PCT Filed: Dec. 23, 2021
(86) PCT No.: PCT/CN2021/140983

§ 371 Date: Aug. 4, 2023

(30) Foreign Application Priority Data
Feb. 5, 2021 (CN) 202110164804.5

(51) Int. Cl. A61K 9/00; A61K 38/26

(52) U.S. Cl. CPC A61K 9/0075; A61K 38/26

(57) ABSTRACT

The present disclosure relates to an inhalable pharmaceutical powder formulation and a preparation method therefor. Specifically, the inhalable pharmaceutical powder formulation of the present disclosure comprises semaglutide and pharmaceutically acceptable excipients, wherein a mass median aerodynamic diameter of the pharmaceutical powder formulation is 0.5 μm-10 μm.

### TECHNICAL FIELD

The present disclosure relates to an inhalable pharmaceutical powder formulation and a preparation method therefor.

### BACKGROUND ART

In recent years, as a non-communicable chronic disease that seriously affects human health and quality of life, diabetes and its complications have become a health issue of global concern, making governments all over the world pay great attention to the research and development of drugs for treating diabetes. For many pharmaceutical manufacturers, conquering diabetes as soon as possible is not only the social responsibility, but also a pursuit of substantial economic benefits. The prevalence rate of diabetes is increasing rapidly and shows a younger trend. One of the important reasons is obesity caused by unhealthy lifestyle. Type II diabetes is a common endocrine and metabolic disease, and obesity is currently considered to be the main risk factor for diabetes. Clinically, obese patients with type II diabetes have the “three high” characteristics of hyperglycemia, hyperlipidemia and hypertension. Among various complex factors that induce diabetes, obesity is the most dangerous signal. To prevent and treat diabetes, it is necessary to control body weight.

Semaglutide is a new long-acting glucagon-like peptide-1 (GLP-1) analog that stimulates insulin secretion and inhibits glucagon secretion in a glucose concentration-dependent mechanism, which can significantly improve blood glucose level in patients with type II diabetes, with a low risk of hypoglycemia. In addition, semaglutide is able to induce significant weight loss by reducing appetite and reducing food intake. Semaglutide is developed by Novo Nordisk, and its injectable and oral formulations are currently approved for marketing.

However, injectable and oral formulations of semaglutide have a relatively low bioavailability (only about 1%), and are more likely to cause gastrointestinal adverse reactions, such as nausea and vomiting. In addition, injectable formulations have high requirements for transportation and storage conditions and must be maintained under cold chain conditions; moreover, the frequency of medication is subcutaneous injection once a week, which is very painful for diabetic patients who need long-term treatment or even lifelong treatment, leading to not only poor compliance, but also susceptibility to infection, and bringing physical and psychological burdens to patients. The medication requirements for oral formulations are very strict: oral formulations must be swallowed with <100 ml of plain water at empty stomach, and within half an hour after medication, no food, beverages, or other medications can be consumed, which compromise the convenience compared with other oral medicines, thereby affecting the compliance of patients. In addition, the long-term safety of oral formulations needs to be further verified by large-scale studies, such as CVOT studies.

### SUMMARY OF THE INVENTION

According to one embodiment of the present disclosure, the present disclosure may provide an inhalable pharmaceutical powder formulation, comprising semaglutide and pharmaceutically acceptable excipients, wherein a mass median aerodynamic diameter of the pharmaceutical powder formulation is 0.5 μm-10 μm.

According to one embodiment of the present disclosure, the present disclosure may provide a method for preparing the pharmaceutical powder formulation as disclosed in the present invention, the method comprises the following steps:
(1) mixing semaglutide, a pharmaceutically acceptable excipient and purified water to obtain a precursor solution; and
(2) performing spray freeze drying on the precursor solution obtained in step (1).

### BRIEF DESCRIPTION OF THE DRAWINGS

FIG. 1 shows the ACI measurement results of the powder formulation of comparative example 1.

FIG. 2 shows the ACI measurement results of the powder formulation of comparative example 2.

FIG. 3 shows the NGI measurement results of the powder formulation of example 6.

FIG. 4 shows the scanning electron microscope image of the powder formulation of example 7.

FIG. 5 shows the NGI measurement results of the powder formulation of example 7.

FIG. 6 shows the scanning electron micrograph of the powder formulation of example 8.

FIG. 7 shows the NGI measurement results of the powder formulation of example 8.

FIG. 8 shows the scanning electron micrograph of the powder formulation of example 9.

FIG. 9 shows the NGI measurement results of the powder formulation of example 9.

FIG. 10 shows the NGI measurement results of the powder formulation of example 10.

FIG. 11 shows the scanning electron micrograph of the powder formulation of example 11.

FIG. 12 shows the NGI measurement results of the powder formulation of example 11.

FIG. 13 shows the scanning electron micrograph of the powder formulation of example 12.

FIG. 14 shows the NGI measurement results of the powder formulation of example 12.

FIG. 15 shows the scanning electron micrograph of the powder formulation of example 13.

FIG. 16 shows the NGI measurement results of the powder formulation of example 13.

### DETAILED DESCRIPTION OF EMBODIMENTS

Unless otherwise indicated, all numbers representing content, concentration, ratio, weight, particle diameter, percentage, technical effect, and so forth as used in the description and claims are to be understood as being modified in any case by the term “about” or “approximately”. Accordingly, unless indicated to the contrary, numerical parameters as set forth in the following description and attached claims are approximations. Unless otherwise indicated, terms as used herein have the meanings commonly understood by those skilled in the art. For those skilled in the art, each numerical parameter may vary depending upon the desired properties and effects sought to be obtained by the present disclosure and should be construed in light of the significant figures of digits and ordinary rounding techniques or in a manner understood by those skilled in the art.

Although the broad range of the numerical values and the parameters which are approximations of the present disclosure are as set forth herein, the numerical values as set forth in the specific examples are given as precisely as possible. However, any numerical value inherently contains certain errors, which are inevitably caused by the standard deviation found in their respective test measurements. Each numerical range given in the description will include every narrower numerical range that falls within such broader numerical range, as if such narrower numerical ranges were all explicitly written herein.

As used herein, the expression “A and/or B” includes three cases: (1) A; (2) B; and (3) A and B. The expression “A, B and/or C” includes seven cases: (1) A; (2) B; (3) C; (4) A and B; (5) A and C; (6) B and C; and (7) A, B and C. The meaning of similar expressions may be in this similar way.

An inhalable pharmaceutical powder formulation is a special dosage form for pulmonary administration, and by means of local administration, the pharmaceutical powder can rapidly and directly enter the lungs to exert its drug efficacy, thereby reducing the administration dosage and improving the drug efficacy.

As used herein, the term “aerodynamic diameter (Da)”, also referred to as aerodynamic equivalent diameter, is a hypothetical diameter (particle diameter) that expresses the motion of the particles. It is defined by W. Stober as the diameter at which a sphere with a unit density (ρ₀=1 g/cm³) reaches the same final settling velocity (Vs) as an actual particle when performing low Reynolds numbers motion in still air. That is, the actual particle diameter is replaced with an equivalence diameter (or equivalent diameter) with the same aerodynamic characteristics. Generally, the actual particle diameter and density cannot be measured; however, the aerodynamic diameter can be measured directly by a dynamic method, so that the particle diameter of particles with different shapes, densities and optical and electrical properties can be measured uniformly. The aerodynamic diameter can be calculated with reference to the following method: the particle diameter (volume particle diameter) Dv of a powder sample is measured by a laser particle size analyzer, and the aerodynamic diameter Da is calculated according to Da=(ρ/ρ₁)^{1/2}×Dv, in which, p is the density of the particle, and ρ₁=1 g/cm³ Dv is the average particle diameter of the particle. The ρ value can be estimated from a tap density, wherein ρ is about 1.26 times the tap density.

As used herein, the term “mass median aerodynamic diameter” or “MMAD” refers to: a particle diameter when the total mass of particles having various sizes smaller than a certain aerodynamic diameter in particulate matters accounts for 50% of the mass of all the particulate matters (i.e., the sum of the masses of all particles having different sizes).

As used herein, the term “fine particle fraction” or “FPF” refers to a percentage of a dose of a particle having a particle diameter less than or equal to 5 μm in the total delivered dose, calculated as follows:

$$FPF = \frac{FPD}{EmittedDose}$$

in which,
FPD is fine particle dose, i.e., the dose of a particle having a mass median aerodynamic diameter less than or equal to 5 μm, calculated according to the masses of drugs entering each stage of ACI or NGI and the corresponding cut-off particle diameter of each stage at a test flow rate;
and Emitted Dose is the total delivered dose, which refers to the sum of the masses of drugs, excluding capsule residue and device residue, entering each stage of ACI or NGI.

As used herein, the term “acidic amino acid” has the meaning commonly understood in the art, and includes aspartic acid and glutamic acid.

As used herein, the term “alkaline amino acid” has the meaning commonly understood in the art, and includes arginine, lysine and histidine.

As used herein, the term “neutral amino acid” has the meaning commonly understood in the art, and includes glycine, alanine, leucine, isoleucine, valine, cystine, cysteine, methionine, threonine, serine, phenylalanine, tyrosine, tryptophan, proline, methionine and hydroxyproline, as well as asparagine and glutamine.

The present disclosure provides an inhalable powder formulation of semaglutide which does not require cold chain storage, has the advantages of simple and convenient medication method, small particle diameter, high lung delivery efficiency, low dosage and less adverse reactions, and can significantly improve patient compliance with administration.

According to one embodiment of the present disclosure, the present disclosure may provide an inhalable pharmaceutical powder formulation comprising semaglutide and pharmaceutically acceptable excipients, wherein a mass median aerodynamic diameter of the pharmaceutical powder formulation is 0.5 μm-10 μm.

In some embodiments of the present disclosure, the pharmaceutically acceptable excipients are selected from amino acids and/or mannitol.

In some embodiments of the present disclosure, the amino acids may acidic amino acids, neutral amino acids and/or alkaline amino acids. In some embodiments of the present disclosure, the acidic amino acids may be selected from glutamic acid and/or structural analogs thereof. In some embodiments of the present disclosure, the acidic amino acids may be selected from glutamic acid and/or aspartic acid. In some embodiments of the present disclosure, the acidic amino acids may be glutamic acid. In some embodiments of the present disclosure, the alkaline amino acids may be selected from lysine and/or structural analogs thereof. In some embodiments of the present disclosure, the alkaline amino acids may be selected from lysine, arginine and/or histidine. In some embodiments of the present disclosure, the alkaline amino acids may be lysine. In some embodiments of the present disclosure, the neutral amino acids may be selected from glycine, leucine and/or structural analogs thereof. In some embodiments of the present disclosure, the neutral amino acids may be selected from glycine, leucine, alanine, methionine, isoleucine and/or valine. In some embodiments of the present disclosure, the neutral amino acids may be selected from leucine, isoleucine and/or valine. In some embodiments of the present disclosure, the neutral amino acids may be selected from leucine and/or isoleucine. In some embodiments of the present disclosure, the neutral amino acids may be leucine.

In some embodiments of the present disclosure, the amino acids may be selected from glycine, leucine, glutamic acid and/or lysine. In some embodiments of the present disclosure, the amino acids may be selected from leucine, glutamic acid and/or lysine.

In some embodiments of the present disclosure, the pharmaceutically acceptable excipients may be selected from amino acids and/or mannitol; preferably, the pharmaceutically acceptable excipients may be selected from neutral amino acids and/or mannitol; more preferably, the pharmaceutically acceptable excipients are selected from valine, leucine, isoleucine and/or mannitol; more preferably, the pharmaceutically acceptable excipients are selected from leucine and/or mannitol; more preferably, the pharmaceutically acceptable excipient is leucine or mannitol; and more preferably, the pharmaceutically acceptable excipient is leucine.

In some embodiments of the present disclosure, the mass median aerodynamic diameter of the pharmaceutical powder formulation is 0.5 nm-10 nm. In some embodiments of the present disclosure, the mass median aerodynamic diameter of the pharmaceutical powder formulation is 0.5 nm-5 nm; preferably, the mass median aerodynamic diameter of the pharmaceutical powder formulation is 0.5 nm-4 nm; and preferably, the mass median aerodynamic diameter of the pharmaceutical powder formulation is 0.5 nm-3 nm.

In some embodiments of the present disclosure, the weight ratio of semaglutide and the excipients is in a range of 1:50 to 50:1; preferably, the weight ratio of semaglutide and the excipients is in a range of 1:20 to 20:1; more preferably, the weight ratio of semaglutide and the excipients is in a range of 1:10 to 10:1; more preferably, the weight ratio of semaglutide and the excipients is in a range of 1:5 to 5:1; more preferably, the weight ratio of semaglutide and the excipients is in a range of 1:4 to 4:1; more preferably, the weight ratio of semaglutide and the excipients is in a range of 1:3 to 3:1; more preferably, the weight ratio of semaglutide and the excipients is in a range of 1:2 to 2:1; more preferably, the weight ratio of semaglutide and the excipients is in a range of 1:14 to 14:1; more preferably, the weight ratio of semaglutide and the excipients is in a range of 1:4 to 14:1; more preferably, the weight ratio of semaglutide and the excipients is in a range of 1:2 to 14:1; and more preferably, the weight ratio of semaglutide and the excipients is in a range of 1:2 to 4:1.

In some embodiments of the present disclosure, the pharmaceutical powder formulation is obtained by a spray freeze drying process.

The above-mentioned various embodiments and preferences for the pharmaceutical powder formulation of the present disclosure can be combined with each other (as long as they are not inherently contradictory to each other), and the various embodiments formed by the combination are considered as a part of the present disclosure.

According to one embodiment of the present disclosure, the present disclosure may provide a method for preparing the pharmaceutical powder formulation of the present disclosure, the method comprises the following steps:
(1) mixing semaglutide, pharmaceutically acceptable excipients and purified water to obtain precursor solution;
(2) performing spray freeze drying on the precursor solution obtained in step (1).

In some embodiments of the present disclosure, the precursor solution obtained in step (1) is sprayed into liquid nitrogen or into a spray cooling tower; preferably, the precursor solution obtained in step (1) is sprayed into the spray cooling tower.

In some embodiments of the present disclosure, the sum of the weights of semaglutide and the pharmaceutically acceptable excipients is 1% to 30% of the total weight of the precursor solution; preferably, the sum of the weights of semaglutide and the pharmaceutically acceptable excipients is 1% to 20% of the total weight of the precursor solution; preferably, the sum of the weights of semaglutide and the pharmaceutically acceptable excipients is 1% to 15% of the total weight of the precursor solution; preferably, the sum of the weights of semaglutide and the pharmaceutically acceptable excipients is 1% to 10% of the total weight of the precursor solution; preferably, the sum of the weights of semaglutide and the pharmaceutically acceptable excipients is 1% to 9% of the total weight of the precursor solution; preferably, the sum of the weights of semaglutide and the pharmaceutically acceptable excipients is 1% to 8% of the total weight of the precursor solution; preferably, the sum of the weights of semaglutide and the pharmaceutically acceptable excipients is 1% to 7% of the total weight of the precursor solution; preferably, the sum of the weights of semaglutide and the pharmaceutically acceptable excipients is 1% to 6% of the total weight of the precursor solution; preferably, the sum of the weights of semaglutide and the pharmaceutically acceptable excipients is 1% to 5% of the total weight of the precursor solution; preferably, the sum of the weights of semaglutide and the pharmaceutically acceptable excipients is 3% to 10% of the total weight of the precursor solution; and preferably, the sum of the weights of semaglutide and the pharmaceutically acceptable excipients is 3% to 10% of the total weight of the precursor solution.

The above-mentioned various embodiments and preferences for the method for preparing the pharmaceutical powder formulation of the present disclosure can be combined with each other (as long as they are not inherently contradictory to each other), and the various embodiments formed by the combination are considered as a part of the present disclosure.

More specifically, the present disclosure further provides the following embodiments:

### Embodiment 1

An inhalable pharmaceutical powder formulation, comprising semaglutide and pharmaceutically acceptable excipients, wherein a mass median aerodynamic diameter of the pharmaceutical powder formulation is 0.5 μm-10 μm.

### Embodiment 2

The pharmaceutical powder formulation of embodiment 1, wherein the pharmaceutically acceptable excipients are selected from amino acids and/or mannitol.

### Embodiment 3

The pharmaceutical powder formulation of embodiment 1 or 2, wherein the pharmaceutically acceptable excipients are selected from neutral amino acids and/or mannitol.

### Embodiment 4

The pharmaceutical powder formulation of any one of the preceding embodiments, wherein the pharmaceutically acceptable excipients are selected from valine, leucine, isoleucine and/or mannitol.

### Embodiment 5

The pharmaceutical powder formulation of any one of the preceding embodiments, wherein the pharmaceutically acceptable excipients are selected from leucine and/or mannitol.

### Embodiment 6

The pharmaceutical powder formulation of any one of the preceding embodiments, wherein the pharmaceutically acceptable excipient is leucine.

### Embodiment 7

The pharmaceutical powder formulation of any one of the preceding embodiments, wherein the mass median aerodynamic diameter of the pharmaceutical powder formulation is 0.5 μm-5 μm.

### Embodiment 8

The pharmaceutical powder formulation of any one of the preceding embodiments, wherein the mass median aerodynamic diameter of the pharmaceutical powder formulation is 0.5 μm-3 μm.

### Embodiment 9

The pharmaceutical powder formulation of any one of the preceding embodiments, wherein the weight ratio of the semaglutide and the excipients is in a range of 1:10 to 10:1.

### Embodiment 10

The pharmaceutical powder formulation of any one of the preceding embodiments, wherein the weight ratio of the semaglutide and the excipients is in a range of 1:5 to 5:1.

### Embodiment 11

The pharmaceutical powder formulation of any one of the preceding embodiments, wherein the pharmaceutical powder formulation is obtained by a spray freeze drying process.

### Embodiment 12

The pharmaceutical powder formulation of any one of the preceding embodiments, wherein the pharmaceutically acceptable excipients are selected from glycine, leucine, glutamic acid and/or lysine.

### Embodiment 13

The pharmaceutical powder formulation of any one of the preceding embodiments, wherein the pharmaceutically acceptable excipients are selected from leucine, glutamic acid and/or lysine.

### Embodiment 14

The pharmaceutical powder formulation of any one of the preceding embodiments, wherein the mass median aerodynamic diameter of the pharmaceutical powder formulation is 0.5 μm-4 μm.

### Embodiment 15

The pharmaceutical powder formulation of any one of the preceding embodiments, wherein the weight ratio of the semaglutide and the pharmaceutically acceptable excipients is in a range of 1:14 to 14:1, preferably in a range of 1:4 to 14:1, and more preferably in a range of 1:2 to 14:1.

### Embodiment 16

A method for preparing the pharmaceutical powder formulation of any one of embodiments 1-11, the method comprises the following steps:
(1) mixing semaglutide, pharmaceutically acceptable excipients and purified water to obtain precursor solution;
(2) performing spray freeze drying on the precursor solution obtained in step (1).

### Embodiment 17

The method of embodiment 16, wherein the precursor solution obtained in step (1) is sprayed into a spray cooling tower.

### Embodiment 18

The method of embodiment 16 or 17, wherein the sum of the weights of the semaglutide and the pharmaceutically acceptable excipients is 1% to 30% of the total weight of the precursor solution.

### Embodiment 19

A method for preparing the pharmaceutical powder formulation of any one of embodiments 12-15, the method comprises the following steps:
(1) mixing semaglutide, pharmaceutically acceptable excipients and purified water to obtain a precursor solution;
(2) performing spray freeze drying on the precursor solution obtained in step (1).

### Embodiment 20

the method of embodiment 19, wherein the precursor solution obtained in step (1) is sprayed into a spray cooling tower.

### Embodiment 21

The method of embodiment 19 or 20, wherein the sum of the weights of the semaglutide and the pharmaceutically acceptable excipients is 1% to 30% of the total weight of the precursor solution.

The technical solutions of the present disclosure will be more clearly and explicitly described by way of illustration in combination with examples. It should be understood that these examples are only for illustrative purposes and not intended to limit the scope of protection of the present disclosure. The scope of protection of the present disclosure is only defined by the claims.

### EXAMPLES

### Materials and Methods

In examples, semaglutide used was purchased from Shenzhen JYMed Technology Co., Ltd.; lactose monohydrate used was purchased from DFE Pharma GmbH & Co.KG; trehalose used was purchased from DFE Pharma GmbH & Co.KG; mannitol used was purchased from ROQUETTE, France; glycine used was purchased from Sinopharm Chemical Reagent Co., Ltd.; leucine used was purchased from Aladdin Reagent (Shanghai) Co., Ltd.; glutamic acid used was purchased from Sinopharm Chemical Reagent Co., Ltd.; and lysine used was purchased from Aladdin Reagent (Shanghai) Co., Ltd.

Mass median aerodynamic diameter and fine particle fraction were measured by an Andersen 8-stage cascade impactor (ACI cascade sampler) or a next-generation 8-stage impactor (NGI cascade sampler). The specific operational procedure was as follows: the pharmaceutical powder was filled in a size 3 capsule, and connected to the artificial throat air inhaling end of an impactor using a Breezhaler inhaler device and a device adapter; with the air pumping rate of a pump adjusted to 60 L/min and the pumping time set to 4 s, the capsule was punctured, and inhalation was started, allowing the pharmaceutical powder to enter different stages of the impactor with the air flow; and the pharmaceutical powder in different stages of the impactor were washed with purified water, placed in a volumetric flask and subjected to volumetric dilution, and the samples were taken for content detection of the pharmaceutical powder in different stages of the impactor using high performance liquid chromatography.

The parameters of the spray freeze drying process performed in the spray cooling tower were as follows:
a Spray freezing parameters:

| | Atomizing nozzle | |
|---|---|---|
| Atomizing nozzle | B-290 of BUCHI | |
| Temperature of spray cooling tower | −60° | C. |
| Flow rate of atomizing air | 17 | L/min |
| Injection speed of feed liquid | 5 | mL/min |

b Freeze-drying curve parameters:

| Refrigeration control | Set temperature | Heating-up time | Duration | |
|---|---|---|---|---|
| Stage 1 | −45° C. | 90 min | 90 min | |
| Primary drying | Set temperature | Heating-up time | Duration | Set vacuum |
| Stage 1 | −12° C. | 90 min | 1200 min | 0.02 mbar |
| Stage 2 | 0° C. | 45 min | 300 min | 0.02 mbar |
| Vacuum drying | Set temperature | Heating-up time | Duration | Set vacuum |
| Stage 1 | 40° C. | 60 min | 600 min | 0.02 mbar |

### Comparative Example 1: Preparation of Powder Formulation by Performing Direct Sieving on Bulk Drug Semaglutide

a Formula

Bulk drug semaglutide.

b Process

The bulk drug semaglutide was sieved with a 60-mesh sieve, and the sieved pharmaceutical powder was detected by the Andersen 8-stage cascade impactor.

c Results

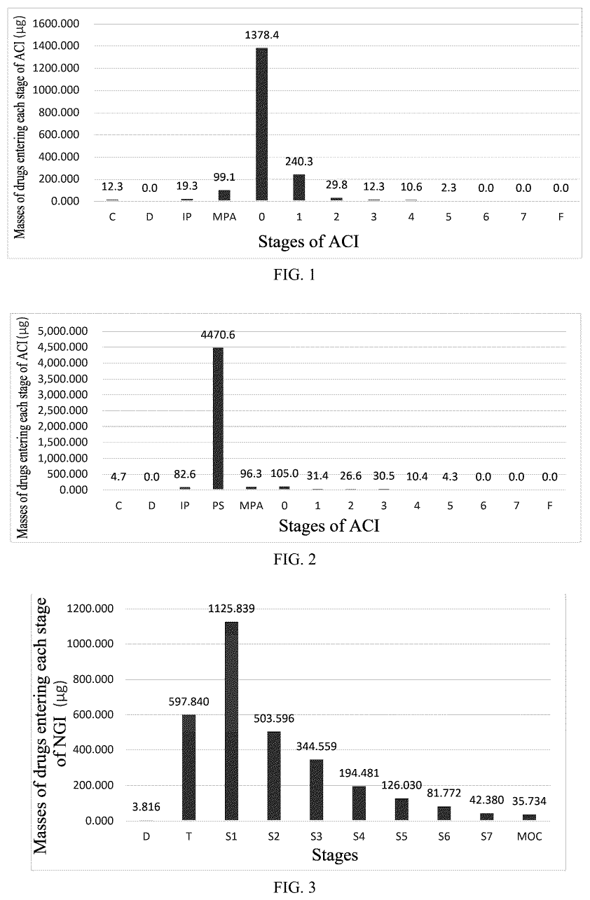

The ACI measurement results were as shown in FIG. 1. As can be seen from FIG. 1, most of the drugs stayed in stage 0. By calculation, the obtained powder formulation had the fine particle fraction of only 3.517%, and the mass median aerodynamic diameter was not applicable here.

### Comparative Example 2: Preparation of Powder Formulation by Performing Low-Energy Mixing on Semaglutide and Lactose Monohydrate

a Formula

| Ingredient | | Proportion (%) | Amount (mg) |
|---|---|---|---|
| Semaglutide | | 20 | 200 |
| Lactose | Respitose SV003 | 70 | 700 |
| monohydrate | Respitose ML006 | 10 | 100 |

b Process

| | | | | | | | Feeding | Mate- | Weight | Param- | Mate- | Weight | Param- | Acqui- |
|---|---|---|---|---|---|---|---|---|---|---|---|---|---|---|
| Mate- | Weight | Param- | Feeding | Mate- | Weight | Param- | sequence | rial | (mg) | eter | rial | (mg) | eter | sition |
| rial | (mg) | eter | sequence | rial | (mg) | eter | Step 3 | | | | Step 4 | | | Step 5 |
| Step 1 | | | Step 2 | | | | (Sieving mixed | | | | (Mixing lactose | | | (Acqui- |
| (Sieving lactose, 11 g) | | | (Mixing lactose, 8 g) | | | | lactose and API, 1 g) | | | | and API, 1 g) | | | sition) |
| SV003 | 9000 | 60- | 1 | Sieved | 2333.3 | 50 rpm, | 1 | Mixed | 266.67 | 60- | Sieved | 1000 | 50 rpm, | Double |
| | | mesh | | SV003 | | 30 min | | lactose | | mesh | mixed | | 30 min | aluminum |
| | | sieve | 2 | Sieved | 500 | | 2 | API | 100 | sieve | powder | | | foil |
| | | | | ML006 | | | | | | | | | | pouch, |
| ML006 | 2000 | | 3 | Sieved | 2333.3 | | 3 | Mixed | 266.67 | | | | | nitrogen- |
| | | | | SV003 | | | | lactose | | | | | | filled |
| | | | 4 | Sieved | 500 | | 4 | API | 100 | | | | | sealed |
| | | | | ML006 | | | | | | | | | | storage. |
| | | | 5 | Sieved | 2333.3 | | 5 | Mixed | 266.67 | | | | | |
| | | | | SV003 | | | | lactose | | | | | | |

c Results
1) Mixing uniformity of the mixed powder

| Sampling points | 1 | 2 | 3 | 4 | 5 | Mean value (%) | RSD (%) |
|---|---|---|---|---|---|---|---|
| Content of the mixed powder (%) | 100.76 | 126.50 | 78.20 | 57.40 | 86.31 | 89.84 | 28.72 |

From the data on the mixing uniformity, the mixed powder was not mixed uniformly.
2) ACI measurement results

The ACI measurement results were as shown in FIG. 2. As can be seen from FIG. 2, most of the drugs stayed in a pre-separator (PS). By calculation, the obtained powder formulation had the fine particle fraction of only 3.517%, and the mass median aerodynamic diameter was not applicable here.

### Comparative Example 3: Preparation of Powder Formulation by Performing Spray Drying on Semaglutide and Lactose Monohydrate

Spray drying requires a high temperature (60° C.-180° C.) to dry bulk drug solution, but the bulk drug semaglutide cannot tolerate such high temperature. In addition, semaglutide is a high-value active ingredient, but the yield of semaglutide by a spray drying process is low. Taking the above factors together, performing spray drying on semaglutide and lactose monohydrate is not suitable for the preparation of an inhalable powder formulation.

### Comparative Example 4: Preparation of Powder Formulation by Performing Spray Freeze Drying on Semaglutide and Lactose Monohydrate

It was found experimentally that lactose monohydrate and semaglutide were subjected to a Maillard reaction, forming new impurities.

### Comparative Example 5: Preparation of Powder Formulation by Performing Spray Freeze Drying on Semaglutide and Trehalose

It was found experimentally that the powder formulation obtained by spray freeze drying with trehalose as an excipient had serious moisture absorption and poor stability; in addition, trehalose and semaglutide were subjected to a Maillard reaction, forming new impurities.

### Example 6: Preparation of Powder Formulation by Performing Spray Freeze Drying on Semaglutide and Mannitol

a Formula

Semaglutide and mannitol 1:2 solution with solid content (w %) of 12.5%

| | | | | Addition of |
|---|---|---|---|---|
| Solid content of | Semaglu- | Semaglu- | | purified |
| the precursor | tide:excipient | tide | Mannitol | water to |
| solution (%) | (w/w) | (g) | (g) | (g) |
| 12.5 | 1:2 | 0.4165 | 0.8361 | 10 |

b Process

The semaglutide and mannitol 1:2 solution with a solid content (w %) of 12.5% was sprayed into liquid nitrogen with the flow rate of an atomizing air adjusted to 60 mm Hg and the feeding speed of the pharmaceutical solution to 15% using atomizing nozzle B-290 of a BUCHI spray dryer, and then transferred to a freeze dryer for freeze drying.

c Results

1) NGI measurement results:

The NGI measurement results were as shown in FIG. 3. By calculation, the obtained powder formulation had the fine particle fraction of 24.107% and the mass median aerodynamic diameter of 9.037 μm.

### Example 7: Preparation of Powder Formulation by Performing Spray Freeze Drying on Semaglutide and Mannitol

a Formula

Semaglutide and mannitol 1:2 solution with solid content (w %) of 5%

| | | | | Addition of |
|---|---|---|---|---|
| Solid content of | Semaglu- | Semaglu- | | purified |
| the precursor | tide:excipient | tide | Mannitol | water to |
| solution (%) | (w/w) | (g) | (g) | (g) |
| 5 | 1:2 | 0.5 | 1.0 | 30 |

b Process

The semaglutide and mannitol 1:2 solution with a solid content (w %) of 5% was sprayed into a spray cooling tower at −60° C. with the flow rate of an atomizing air adjusted to 17 L/min and the feeding speed of a pharmaceutical solution to 5 mL/min using atomizing nozzle B-290 of a BUCHI spray dryer, and then transferred to a freeze dryer for freeze drying.

c Results
1) Scanning electron microscope results:

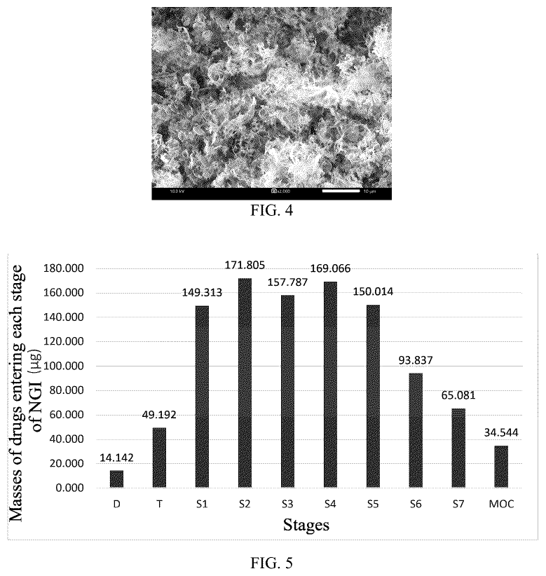

The obtained powder formulation was scanned by a high resolution field emission scanning electron microscope after spray-gold treatment, and the obtained scanning electron micrograph was as shown in FIG. 4. As can be seen from FIG. 4, the powder formulation obtained after the spray freezing and drying of mannitol and semaglutide in the spray cooling tower was flocculent.
2) NGI measurement results:

The NGI measurement results were as shown in FIG. 5. By calculation, the obtained powder formulation had the fine particle fraction of 68.125% and the mass median aerodynamic diameter of 2.678 μm.

### Example 8: Preparation of Powder Formulation by Performing Spray Freeze Drying on Semaglutide and Leucine

a Formula

Semaglutide and leucine 1:2 solution with solid content (w %) of 3%

| | | | | Addition of |
|---|---|---|---|---|
| Solid content of | Semaglu- | Semaglu- | | purified |
| the precursor | tide:excipient | tide | Leucine | water to |
| solution (%) | (w/w) | (g) | (g) | (g) |
| 3 | 1:2 | 0.3 | 0.6 | 30 |

b Process

The semaglutide and leucine 1:2 solution with a solid content (w %) of 3% was sprayed into a spray cooling tower at −60° C. with the flow rate of an atomizing air adjusted to 17 L/min and the feeding speed of the pharmaceutical solution to 5 mL/min using atomizing nozzle B-290 of a BUCHI spray dryer, and then transferred to a freeze dryer for freeze drying.

c Results
1) Scanning electron microscope results:

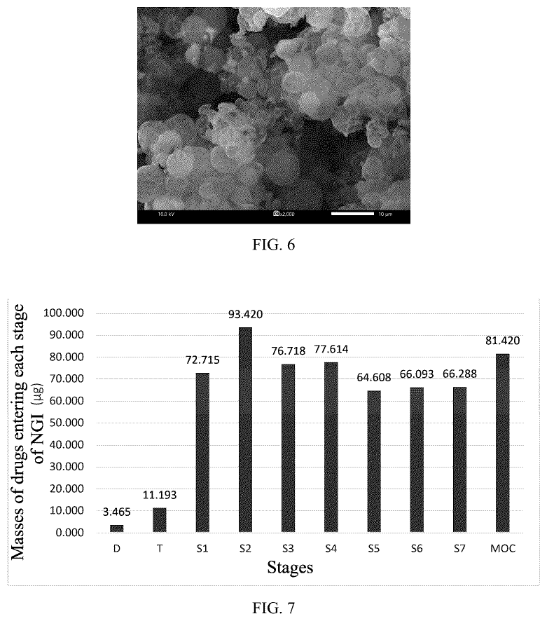

The obtained powder formulation was scanned by a high resolution field emission scanning electron microscope after spray-gold treatment, and the obtained scanning electron micrography was as shown in FIG. 6. As can be seen from FIG. 6, the powder formulation obtained after the spray freezing and drying of leucine and semaglutide in a spray cooling tower was spherical.
2) NGI measurement results:

The NGI measurement results were as shown in FIG. 7. By calculation, the obtained powder formulation had the fine particle fraction of 74.476%, and the mass median aerodynamic diameter of 1.915 μm.

### Example 9: Preparation of Powder Formulation by Performing Spray Freeze Drying on Semaglutide and Leucine

a Formula

Semaglutide and leucine 2:1 solution with solid content (w %) of 6%

| | | | | Addition of |
|---|---|---|---|---|
| Solid content of | Semaglu- | Semaglu- | | purified |
| the precursor | tide:excipient | tide | Leucine | water to |
| solution (%) | (w/w) | (g) | (g) | (g) |
| 6 | 2:1 | 2.4 | 1.2 | 60 |

b Process

The semaglutide and leucine 2:1 solution with a solid content (w %) of 6% was sprayed into a spray cooling tower at −60° C. with the flow rate of an atomizing air adjusted to 17 L/min and the feeding speed of the pharmaceutical solution to 5 mL/min using atomizing nozzle B-290 of a BUCHI spray dryer, and then transferred to a freeze dryer for freeze drying.

c Results
1) Scanning electron microscope results:

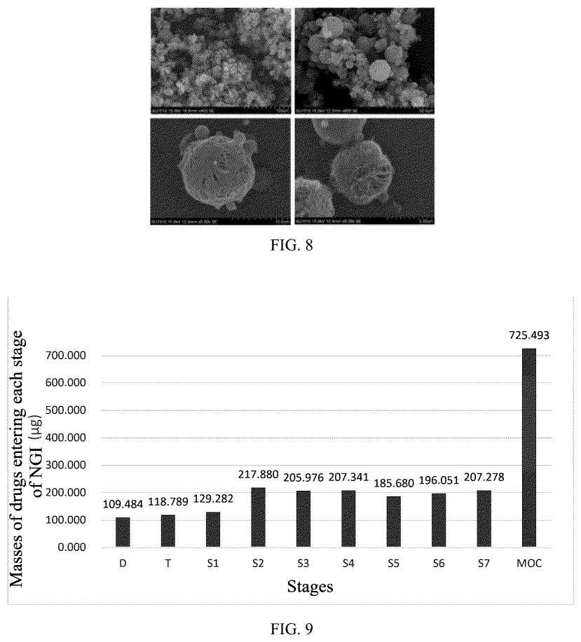

The obtained powder formulation was scanned by a high resolution field emission scanning electron microscope after spray-gold treatment, and the obtained scanning electron micrography was as shown in FIG. 8. As can be seen from FIG. 8, the powder formulation obtained after the spray freezing and drying of leucine and semaglutide in a spray cooling tower was a spherical and porous particle.
2) NGI measurement results:

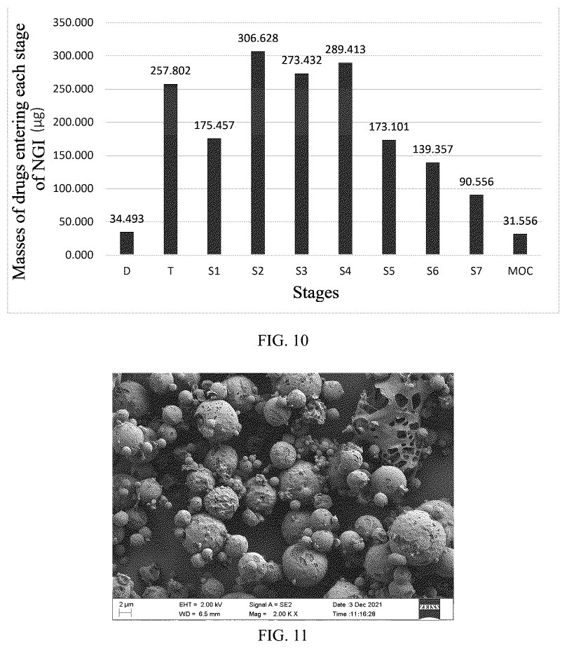

The NGI measurement results were as shown in FIG. 9. By calculation, the obtained powder formulation had the fine particle fraction of 81.23%, and the mass median aerodynamic diameter of 0.732 μm.
3) Geometric dimension results:

| D10 (μm) | D50 (μm) | D90 (μm) |
|---|---|---|
| 5.60 | 12.88 | 26.11 |

The geometric dimension was measured by a Sympatec laser particle size analyzer, with R3 lens selected, a dispersing pressure of 2-3 bar, and a feeding rate of 60%. The powder had a measured physical geometrical dimension D50 of 12.88 μm.

### Example 10: Preparation of Powder Formulation by Performing Spray Freeze Drying on Semaglutide and Leucine

a Formula

Semaglutide and leucine 4:1 solution with solid content (w %) of 10%

| | | | | Addition of |
|---|---|---|---|---|
| Solid content of | Semaglu- | Semaglu- | | purified |
| the precursor | tide:excipient | tide | Leucine | water to |
| solution (%) | (w/w) | (g) | (g) | (g) |
| 10 | 4:1 | 4.8 | 1.2 | 60 |

b Process

The semaglutide and leucine 4:1 solution with a solid content (w %) of 10% was sprayed into a spray cooling tower at −60° C. with the flow rate of an atomizing air adjusted to 17 L/min and the feeding speed of the pharmaceutical solution to 5 mL/min using atomizing nozzle B-290 of a BUCHI spray dryer, and then transferred to a freeze dryer for freeze drying.

c Results
1) Specific surface area results:

The specific surface areas of the bulk drug semaglutide, excipient leucine and powder formulation were respectively detected by an inverse gas chromatography-surface energy analyzer. The specific surface area results were as follows:

| Sample name | Specific surface area (m²/g) |
|---|---|
| Powder formulation | 32.703 |
| Bulk drug semaglutide | 1.816 |
| Excipient leucine | 0.506 |

The specific surface area of the powder formulation prepared in example 10 was 32.703 m²/g, which was much larger than the specific surface area of the bulk drug semaglutide (1.816 m²/g) and the specific surface area of the excipient leucine (0.506 m²/g).
2) NGI measurement results:

The NGI measurement results were as shown in FIG. 10. By calculation, the obtained powder formulation had the fine particle fraction of 61.59%, and the mass median aerodynamic diameter of 2.893 μm.

### Example 11: Preparation of Powder Formulation by Performing Spray Freeze Dryin2 on Semaglutide and Glutamic Acid

a Formula

Semaglutide and glutamic acid 14:1 solution with solid content (w %) of 10%

| | | | | Addition of |
|---|---|---|---|---|
| Solid content of | Semaglu- | Semaglu- | Glutamic | purified |
| the precursor | tide:excipient | tide | acid | water to |
| solution (%) | (w/w) | (g) | (g) | (g) |
| 10 | 14:1 | 2.8 | 0.2037 | 30 |

b Process

The semaglutide and glutamic acid 14:1 solution with a solid content (w %) of 10% was sprayed into a spray cooling tower at −60° C. with the flow rate of an atomizing air adjusted to 17 L/min and the feeding speed of the pharmaceutical solution to 5 mL/min using atomizing nozzle B-290 of a BUCHI spray dryer, and then transferred to a freeze dryer for freeze drying.

c Results
1) Scanning electron microscope results:

The obtained powder formulation was scanned by a high resolution field emission scanning electron microscope after spray-gold treatment, and the obtained scanning electron micrography was as shown in FIG. 11. As can be seen from FIG. 11, the powder formulation obtained after the spray freezing and drying of glutamic acid and semaglutide in a spray cooling tower was a spherical and porous particle.
2) NGI measurement results:

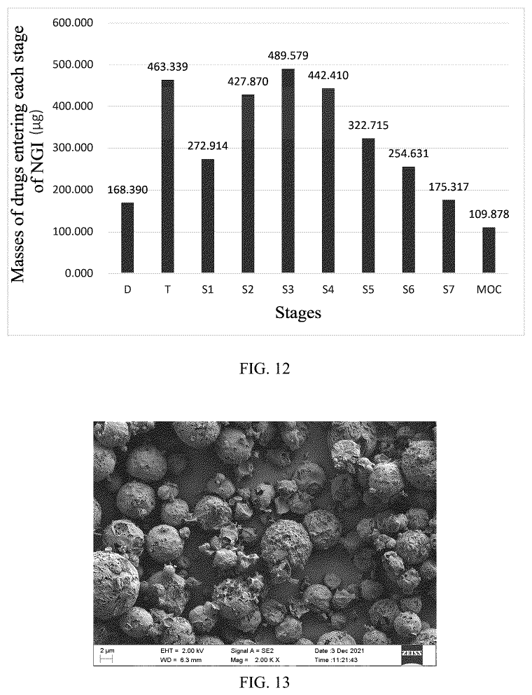

The NGI measurement results were as shown in FIG. 12. By calculation, the obtained powder formulation had the fine particle fraction of 64.09%, and the mass median aerodynamic diameter of 2.637 μm.

### Example 12: Preparation of Powder Formulation by Performing Spray Freeze Drying on Semaglutide and Lysine

a Formula

Semaglutide and lysine 4:1 solution with solid content (w %) of 10%

| | | | | Addition of |
|---|---|---|---|---|
| Solid content of | Semaglu- | Semaglu- | | purified |
| the precursor | tide:excipient | tide | Lysine | water to |
| solution (%) | (w/w) | (g) | (g) | (g) |
| 10 | 4:1 | 2.4 | 0.60 | 30 |

b Process

The semaglutide and lysine 4:1 solution with a solid content (w %) of 10% was sprayed into a spray cooling tower at −60° C. with the flow rate of an atomizing air adjusted to 17 L/min and the feeding speed of the pharmaceutical solution to 5 mL/min using atomizing nozzle B-290 of a BUCHI spray dryer, and then transferred to a freeze dryer for freeze drying.

c Results
1) Scanning electron microscope results:

The obtained powder formulation was scanned by a high resolution field emission scanning electron microscope after spray-gold treatment, and the obtained scanning electron micrography was as shown in FIG. 13. As can be seen from FIG. 13, the powder formulation obtained after the spray freezing and drying of lysine and semaglutide in a spray cooling tower was a spherical and porous particle.
2) NGI measurement results:

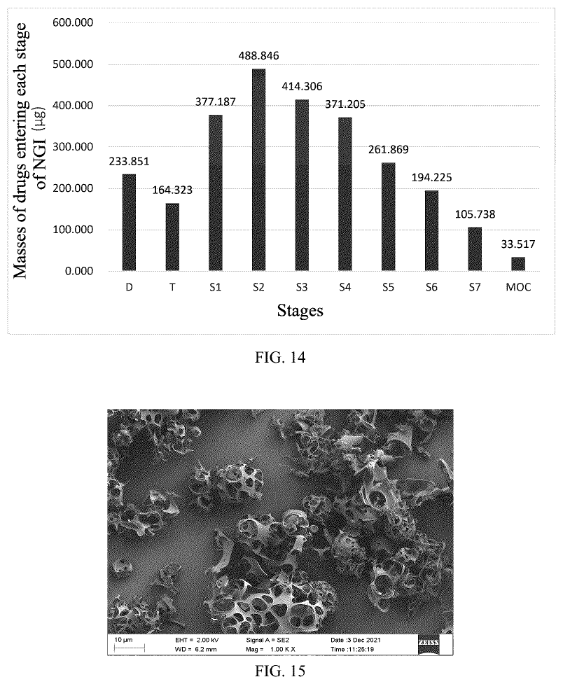

The NGI measurement results were as shown in FIG. 14. By calculation, the obtained powder formulation had the fine particle fraction of 61.79%, and the mass median aerodynamic diameter of 3.351 μm.

### Example 13: Preparation of Powder Formulation by Performing Spray Freeze Drying on Semaglutide and Glycine

a Formula

Semaglutide and glycine 4:1 solution with solid content (w %) of 10%

| | | | | Addition of |
|---|---|---|---|---|
| Solid content of | Semaglu- | Semaglu- | | purified |
| the precursor | tide:excipient | tide | Glycine | water to |
| solution (%) | (w/w) | (g) | (g) | (g) |
| 10 | 4:1 | 2.4 | 0.60 | 30 |

b Process

The semaglutide and glycine 4:1 solution with a solid content (w %) of 10% was sprayed into a spray cooling tower at −60° C. with the flow rate of an atomizing air adjusted to 17 L/min and the feeding speed of the pharmaceutical solution to 5 mL/min using atomizing nozzle B-290 of a BUCHI spray dryer, and then transferred to a freeze dryer for freeze drying.

c Results
1) Scanning electron microscope results:

The obtained powder formulation was scanned by a high resolution field emission scanning electron microscope after spray-gold treatment, and the obtained scanning electron micrography was as shown in FIG. 15. As can be seen from FIG. 15, the powder formulation obtained after the spray freezing and drying of glycine and semaglutide in a spray cooling tower was a spherical and porous particle.

2) NGI measurement results:

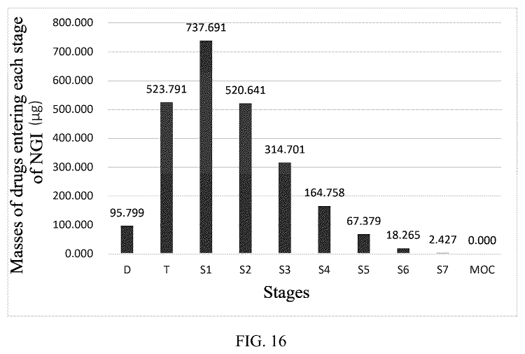

The NGI measurement results were as shown in FIG. 16. By calculation, the obtained powder formulation had the fine particle fraction of 28.19%, and the mass median aerodynamic diameter of 6.631 μm.

1. An inhalable pharmaceutical powder formulation, comprising semaglutide and pharmaceutically acceptable excipients, wherein a mass median aerodynamic diameter of the pharmaceutical powder formulation is 0.5 μm-10 μm.

2. The pharmaceutical powder formulation of claim 1, wherein the pharmaceutically acceptable excipients are amino acids and/or mannitol.

3. The pharmaceutical powder formulation of claim 2, wherein the pharmaceutically acceptable excipients are neutral amino acids and/or mannitol.

4. The pharmaceutical powder formulation of claim 3, wherein the pharmaceutically acceptable excipients are selected from valine, leucine, isoleucine and mannitol.

5. The pharmaceutical powder formulation of claim 4, wherein the pharmaceutically acceptable excipients are leucine and/or mannitol.

6. The pharmaceutical powder formulation of claim 5, wherein the pharmaceutically acceptable excipient is leucine.

7. The pharmaceutical powder formulation of claim 1, wherein the mass median aerodynamic diameter of the pharmaceutical powder formulation is 0.5 μm-5 μm.

8. The pharmaceutical powder formulation of claim 1, wherein the mass median aerodynamic diameter of the pharmaceutical powder formulation is 0.5 μm-3 μm.

9. The pharmaceutical powder formulation of claim 1, wherein the weight ratio of the semaglutide and the excipients is in a range of 1:10 to 10:1.

10. The pharmaceutical powder formulation of claim 1, wherein the weight ratio of the semaglutide and the excipients is in a range of 1:5 to 5:1.

11. The pharmaceutical powder formulation of claim 1, wherein the pharmaceutical powder formulation is obtained by a spray freeze drying process.

12. The pharmaceutical powder formulation of claim 1, wherein the pharmaceutically acceptable excipients are selected from glycine, leucine, glutamic acid and lysine.

13. The pharmaceutical powder formulation of claim 1, wherein the pharmaceutically acceptable excipients are selected from leucine, glutamic acid and lysine.

14. The pharmaceutical powder formulation of claim 1, wherein the mass median aerodynamic diameter of the pharmaceutical powder formulation is 0.5 μm-4 μm.

15. The pharmaceutical powder formulation of claim 1, wherein the weight ratio of the semaglutide and the pharmaceutically acceptable excipients is in a range of 1:14 to 14:1.

16. A method for preparing the pharmaceutical powder formulation of claim 1, the method comprises the following steps:
(1) mixing semaglutide, pharmaceutically acceptable excipients and purified water to obtain precursor solution;
(2) performing spray freeze drying on the precursor solution obtained in step (1).

17. The method of claim 16, wherein the precursor solution obtained in step (1) is sprayed into a spray cooling tower.

18. The method of claim 16, wherein the sum of the weights of the semaglutide and the pharmaceutically acceptable excipients is 1% to 30% of the total weight of the precursor solution.

19-21. (canceled)

22. The pharmaceutical powder formulation of claim 1, wherein the weight ratio of the semaglutide and the pharmaceutically acceptable excipients is in a range of 1:4 to 14:1.

23. The pharmaceutical powder formulation of claim 1, wherein the weight ratio of the semaglutide and the pharmaceutically acceptable excipients is in a range of 1:2 to 14:1.
